(19) United States
(12) Patent Application Publication
(10) Pub. No.: US 2009/0054898 A1
(43) Pub. Date: Feb. 26, 2009

(54) Articulating Shaper

(76) Inventor: Joe Gleason, Eagan, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 12/056,025

(22) Filed: Mar. 26, 2008

Related U.S. Application Data
(60) Provisional application No. 60/919,983, filed on Mar. 26, 2007.

(51) Int. Cl. A61B 17/00

(52) U.S. Cl. 606/79; 606/84

(57) ABSTRACT

A shaper for reaming tissue includes an articulating head operably connected to a shaft. Incremental deployment allows for a unique utility in shaping a cavity suited to the particular morphology, that is, a more customizable cavity shape may be achieved.

### RELATED APPLICATION

This application claims the benefit of U.S. Provisional Application No. 60/919,983 filed Mar. 26, 2007, which is hereby fully incorporated herein by reference.

### FIELD OF THE INVENTION

This invention relates to an apparatus and method for removing, reaming, debriding and/or resecting tissue and/or bone. In particular, the present invention is directed to a shaper that may be incrementally deployed to create a variety of cavity shapes. The apparatus and method of the present invention may be especially useful in medical procedures such as orthopedic surgery.

### BACKGROUND

Medical procedures involving the removal of tissue from a region of a body are well known in the art. The present invention may be particularly useful in spine surgery. A variety of tools are available for surgeons to remove spinal disc tissue and/or bony tissue during surgery in the spinal region.

Pituitary rongeurs and curettes are the most frequently used instruments to remove tissue. Some examples of these instruments are described in the following U.S. Patents: U.S. Pat. Nos.: 6,200,320 to Michelson; 6,142,997 to Michelson; 5,961,531 to Weber et al.; 5,766,177 to Lucas-Dean et al.; 5,653,713 to Michelson; 5,484,441 to Koros et al.; 5,451,227 to Michelson; 5,312,407 to Carter; 5,026,375 to Linovitz et al. 5,061,269 to Muller; 4,990,148 to Worrick, III et al.; 4,777,948 to Wright; 4,733,663 to Farely; 4,722,338 to Wright et al.; 3,902,498 to Niederer; 3,628,524 to Jamshidi and 2,984,241 to Carlson.

The use of rongeurs and curettes tends to leave behind fragments of tissue. Further, because these rongeurs and curettes require multiple passes, the operation may be prolonged, possibly leading to increased bleeding and higher infection rates. Many pituitary rongeurs utilize a single cutting blade at the end of a single, unopposed beam. Actuation of the beam, by means of a drive rod, tends to force the distal shaft to move away from the tissue being cut. An open section in the middle of the beam helps reduce this movement, but does not effectively eliminate the unwanted movement.

U.S. Pat. No. 5,445,639 to Kuslich et al., describes an intervertebral reamer used to ream out the interior of a degenerated disc to clean the interbody space. U.S. Pat. No. 6,383,188 to Kuslich et al., discloses an expandable reamer including a pair of opposing blades which have a expanded state and a retracted state. The blades are pivotally positioned at the distal end of a shaft assembly.

U.S. Pat. No. 6,575,978 to Peterson et al., discloses a circumferential resecting reamer tool. The reamer disclosed in the '978 Patent is a multibladed cutting tool that circumferentially reams tissue. The cutting blades sweep through an arc creating a transverse cavity.

U.S. Pat. No. 5,928,239 to Mirza discloses a reamer which has a shaft and a cutting tip attached through a free rotating hinge such that high speed rotation allows the tip to be deflected outwardly to form a cavity. U.S. Pat. No. 5,591,170 to Spievack et al discloses a powered bone saw which inserts its cutting blade through a bored intramedullary canal.

U.S. Pat. Nos. 6,440,138 and 6,863,672 to Reiley et al., describe tools for creating cavities in bone wherein the tool includes various cutting tips carried on a shaft. The cutting tips disclosed include a rotatable loop, brush, or blade, a linear cutting blade and an energy transmitter. U.S. Pat. No. 6,923,813 to Phillips et al., discloses tools for creating voids in interior body regions. The tools include several different cutting tips which provide for rotational and translational cutting.

While these various surgical tools are effective in creating cavities within a patient's body, it would be desirable to provide a tool for removal of tissue that is capable of more refined control, particularly of cavity shape, yet is simple to operate.

### SUMMARY OF THE INVENTION

In an embodiment, the present invention comprises a shaper for reaming tissue having a single articulating head operably connected to a shaft. According to one aspect, incremental deployment allows for a unique utility in shaping a cavity suited to the particular morphology, that is, a more customizable cavity shape may be achieved.

In one embodiment of the present invention, an articulating shaper for reaming tissue includes as least one cutting element that is operably connected to the distal end of the shaper. An actuator may be operably engaged to the proximal end of the shaper to incrementally deploy the cutting element to create a desired cavity shape. In an embodiment the shaper may include a handle that deploys the cutting element in a sweeping motion. Thus, the shaper may be used to create virtually any cavity shape by deploying the cutting element incrementally and/or in a sweeping motion.

In one embodiment, an internal rod may be threadably connected to an actuator. The actuator may be turned to deploy the cutting end to an intermediate fixed position without activating the handle.

In another embodiment, the shaper may limit input force to prevent shear force failure of distal mechanisms through the use of clutches. One such clutch may be a ball detent clutch that may employ a spring force multiplier fulcrum arm. The ball detent clutch may include an internal rod connected to a female ball detent groove. The ball detent may act as a slip clutch using a fulcrum to decrease the height and multiply the spring force. The clutch may utilize materials with low yield point and fracture loads to limit input forces. According to one aspect the clutch may use spring force and cam angles to limit input forces. In yet another embodiment, the clutching device may include a spring that determines the break away force.

According to one embodiment of the present invention, the shaper may include a handle that may drive a rod carrier forward such that a pin bears on a pressure limiting clip. In an embodiment, a pressure limiting clip may crack when the pressure at the distal end of the shaper reaches a maximum pressure. When the clip cracks, the pin may rebear against the rod carrier enabling the shaper to be closed and removed. In an embodiment, the pressure limiting clip may be unclipped and flipped around for use on the other side.

In another embodiment, the shaper includes a blade at its distal end. The blade may be pivotally actuated by an offset lever arm. The offset lever arm may be connected to and activated by a spring bar through linear, forward, coaxial movement. The spring bar allows the offset lever arm to pivot away from the axis of movement without the requirement of another hinge point and separate link arm. The blade may be pressure sensitive such that a user may be able to feel feedback on the handle as to what tissue the blade is cutting, such as for example, bone, cartilage, annulus or nucleus.

In an embodiment, the shaper may be activated using a squeezing hand motion to generate articulating angular sweeping cutting blade motion. According to one aspect, an articulating cutting blade may include one pivoting connection point to the instrument shaft and one pivoting activation point.

In an embodiment, a spring bar allows coaxial linear force to transition to non-coaxial angular force without a pivoting connection. According to one aspect, the shaper may include multiple links to increase perpendicular cutting offset. According to another aspect, the shaper may include multiple link controls to pivot a cutting head 90 degrees, and to maintain the 90 degree position with additional perpendicular offsets to instrument access.

In an embodiment where the shaper is used to create a cavity in a disc space, the shaper may generally create a cavity parallel to the endplates. In an embodiment where the shaper is used to create a cavity within a collapsed or wedged vertebral body, the shaper may create a cavity generally parallel to the unfractured, caudal vertebral endplate. In another embodiment the shaper may be rotated or moved longitudinally to create virtually any desired cavity shape. In yet another embodiment, the shaper may be used as a curette with the cutting end in a fixed position.

In another embodiment, the shaper may include a two-stage articulated shaper that may be passed through an entrance hole in a collapsed state and deployed to an expanded state.

### BRIEF DESCRIPTION OF THE DRAWINGS

FIG. 1 is a perspective view of an embodiment of a shaper according to the present disclosure.

FIG. 2 is a perspective view of the various components of an embodiment of a shaper according to the present disclosure.

FIG. 3 is a perspective view of a clutch/force multiplier according to one embodiment.

FIG. 4 is an exploded view of a clutch according to one embodiment.

FIG. 5 is an exploded view of a clutch according to another embodiment.

FIG. 6 is a perspective view of an alternate embodiment of a shaper.

### DETAILED DESCRIPTION

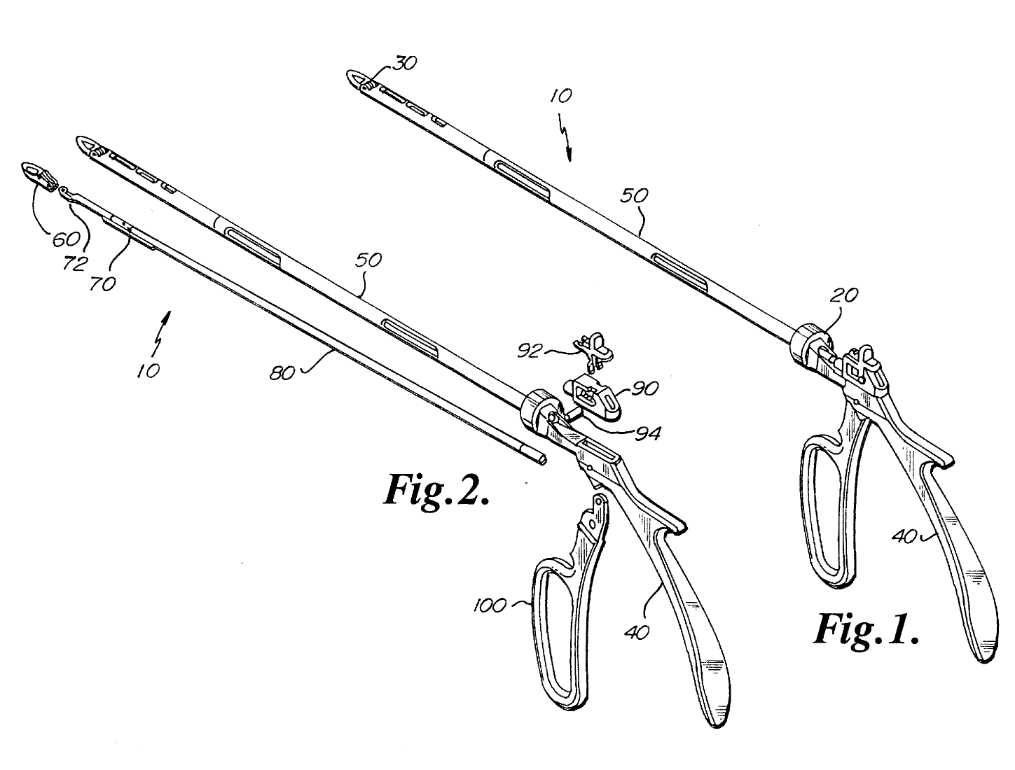

Referring to FIG. 1 there can be seen a shaper 10 according to one aspect of the present invention. Shaper 10 may include a proximal end 20 and a distal end 30. Shaper 10 may further include a handle 40 and a shaft 50. Referring now to FIG. 2, it can be seen that a cutting head 60 may be operably engaged to distal end 30. Cutting head 60 may be a single articulating head. In an embodiment cutting head 60 may be a blade. In one preferred embodiment, cutting head 60 may be operably connected to spring bar 70 and shaft 50. In an embodiment, cutting head 60 may be pivotally connected to spring bar 70 and shaft 50. Spring bar 70 may be operably connected to a driving rod 80 that is operably connected to a clutch box 90. Clutch box 90 may be actuated by a handle lever 100. Spring bar 70 may be adjacent to cutting head 60 such that driving rod 80 may remain concentric to shaft 50. Spring bar 70 may be operably connected to an offset lever arm 72. Offset lever arm 72 may be activated by spring bar 70 in linear, forward coaxial motion. Spring bar 70 allows offset lever arm 72 to pivot away from the axis of movement without requiring another hinge point and separate link arm.

In one embodiment, lever 100 may activate clutch box 90, which in turn moves driving rod 80 forward. Driving rod 80 pushes spring bar 70 forward which articulates cutting head 60. Cutting head 60 may be articulated from a neutral position in line with shaft 50 to a position in the range of about 130 degrees from the center line of shaft 50 and may be fixed in any position there between. In an embodiment, the shaper includes a blade at its distal end. The blade may be pivotally actuated by offset lever arm 72. The blade may be pressure sensitive such that a user may be able to feel feedback on the handle as to what tissue the blade is cutting, such as for example, bone, cartilage, annulus or nucleus.

In an embodiment, the shaper may be activated by squeezing lever 100 to generate articulating angular sweeping cutting blade motion. According to one aspect, an articulating cutting blade may include one pivoting connection point to the instrument shaft and one pivoting activation point.

The articulation of cutting head 60 allows shaper 10 to remove tissue such that a void that is at least twice the width of the blade length may be created. Shaper 10 may be rotated, moved longitudinally or a combination of the two.

In another embodiment, shaper 10 may include an actuator at its proximal end 20. According to one aspect, an actuator may be threadably connected to driving rod 80 such that when the actuator is activated, driving rod 80 may deploy cutting head 60 into incremental fixed positions. An actuator may be used without activating handle lever 100. When cutting head 60 is in a fixed position, shaper 10 may be used as a curette, or scraping tool. Thus, the actuator may be used to limit articulation to a partial stroke.

In one embodiment, shaper 10 may be in the range of about 3 mm to about 7 mm in diameter. When shaper 10 is inserted into a body cavity, cutting head 60 may be in line with shaft 50, allowing shaper 10 to be inserted through a minimally invasive opening such as a small surgical cannula. Because cutting head 60 may be incrementally articulated from a position in line with shaft 50 to a position in the range of about 130 degrees from the center line of shaft 50, virtually any shape cavity may be created.

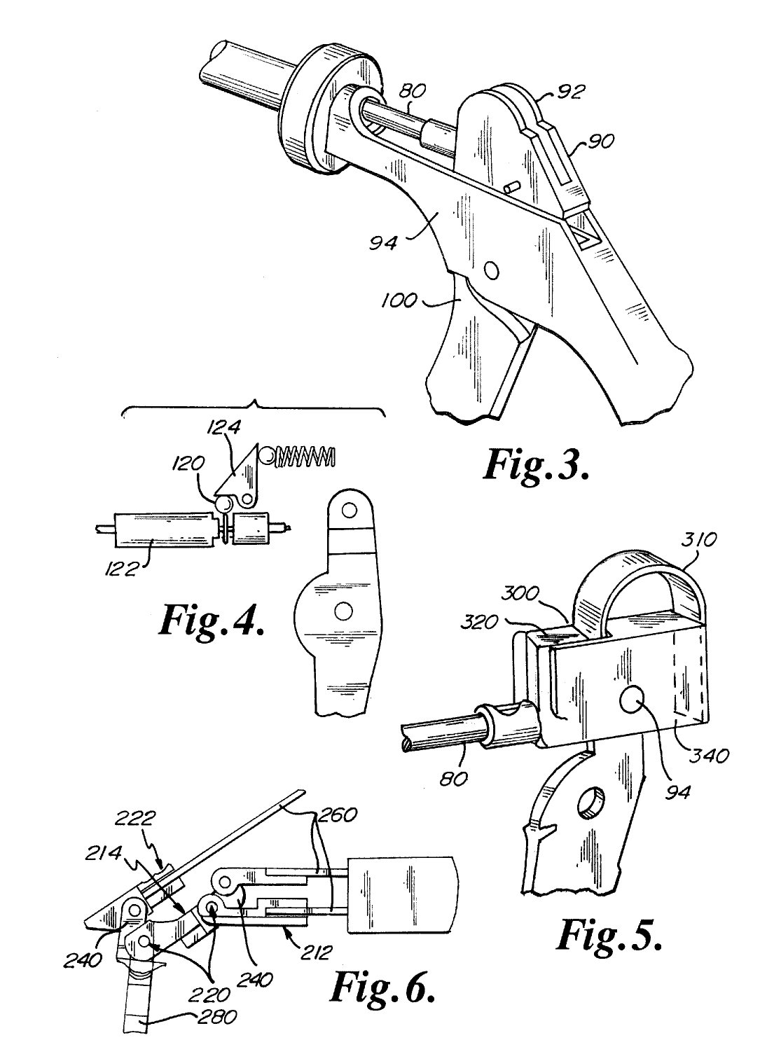

As can be seen in FIG. 3, in an embodiment, a pressure limiting clip 92 may be placed over clutch box 90. Clutch box 90 may further include pin 94. Handle lever 100 may activate clutch box 90 driving clutch box 90 forward such that pin 94 bears on pressure limiting clip 92. In one embodiment, pressure limiting clip 92 may include intentional fracture points such that clip 92 cracks when distal end 30 reaches a maximum pressure in the range of about 30 to 80 psi. A user may be able to hear and feel the crack. Pin 94 may rebear against clutch box 90. A user may then close and remove the instrument. In one embodiment, clip 92 may be unclipped and flipped over for reuse on the uncracked/intact side.

In yet another embodiment, the shaper may limit input force to prevent shear force failure of distal mechanisms through the use of clutches. One such clutch may be a ball detent clutch that may employ a spring force multiplier fulcrum arm. In an embodiment as depicted in FIG. 4, the force multiplier may be a space saving ball detent clutch 120 that may use a pivoting force multiplier 122 to prevent overloading driving rod 80. Clutch 120 may include a ball detent 124 and a fulcrum 126. Ball detent 124 may act as a slip clutch using a fulcrum 126 to decrease the height and multiply the spring force. Driving rod 80 may include a male surface 82 that operably connects to a female surface 112 of the ball detent 124. Fulcrum 126 creates a force multiplier such that smaller diameter springs with restricted force capacity can be used to increase force against the ball for higher disengagement forces while reducing the overall height of the instrument. The clutch may utilize materials with low yield point and fracture loads to limit input forces. According to one aspect the clutch may use spring force and cam angles to limit input forces.

In another embodiment, as depicted in FIG. 5, clutch 300 may include a spring 310 a cam 320 and a clutch body 340. If driving rod 80 is over driven, pin 94 may push cam 320 against spring 310. The amount of force on spring 310 and the angle of cam 320 determine the break away force required.

In use, shaper 10 may be placed generally in the center of a location in a patient's body where a cavity to be created. Cutting head 60 may be articulated to the desired position and used as a scraping tool, and/or cutting head 60 may be used to cyclically sweep out a cavity. A combination of scraping and sweeping may be used to create the desired cavity shape. Further, the articulating cutting head 60 permits shaper 10 to be rotated or moved longitudinally. Shaper 10 may also be placed deeper or shallower in the cavity, to create the desired cavity shape. Because a combination of incremental deployment, sweeping rotating and longitudinal movement may be accomplished with the shaper 10, a customized cavity may be created to comport to the particular morphology being treated.

In an alternate embodiment as depicted in FIG. 6 shaper 200 may be a two-stage articulated shaper that may be passed through an entrance hole in a collapsed state and deployed to an expanded state. In this embodiment, multiple, individually controlled, toggle links in conjunction with flexible control link drivers create a ridged angled shaper. In a collapsed state, the shaper may be inserted through a very small diameter working cannula. In one embodiment, the shaper may be sequentially deployed in two stages. The first stage of deployment may set a desired cutting position of a distal cutting head. The second stage of deployment may drive a cutting head perpendicularly away from the center of the shaper maintaining a predetermined, first stage deployment, cutting head position.

According to one aspect, each stage of deployment may be achieved by individual links including a pivot fulcrum point 220, arm 240, and a flexible driving member 260 operably connected to individual linear drivers. Flexible driving members 260 may be comprised of flexible spring materials including, but not limited to: nitinol, 303 Full Hard stainless steel, 420 stainless steel, and/or 17-4 H900 stainless steel.

Shaper 200 may include a distal cutting head 280 that may be configured to work in a linear scraping action consistent with curettes, or may be configured to work in a sweeping motion. Cutting head 280 may be positioned in a neutral position in line with the center line of the instrument. Cutting head 280 may be deployed to about 90 degrees with about a 20 mm offset of perpendicular from the centerline of the instrument or may be maintained in any position in between. The deployment range is dependent on the number and the length of links.

Shaper 200 may include an anchor link 212 and a deployment link 214. During the second stage of deployment, deployment link 214 may be operably connected to anchor link 212 at the fulcrum position of deployment link 214. Shaper 200 may further include a deployment link activation arm 216 that may be operably connected by a pivot point 218 to a flexible band 260 which may also be operably connected to a proximal instrument linear deployment mechanism. The deployment mechanism may be a screw or cam device or any other suitable deployment mechanism.

The first stage band 222 may deploy cutting head 280 incrementally from a neutral position inline with the shaper to a position in the range of about 90 degrees from neutral. Cutting head 280 may be locked in any position. Cutting head 280 may be deployed by linear band movement to the arm 240. Second stage deployment may be activated by linear motion of the intermediate control band. Deployment may be incremental, and cutting may be done at any stage of deployment. A cavity that is unilateral to the axis of shaper insertion may be created.

This completes the description of the preferred and alternate embodiments of the invention. Those skilled in the art may recognize other equivalents to the specific embodiment described herein which equivalents are intended to be encompassed by the claims attached hereto.

1. A shaper for use in bone and tissue in a mammal comprising:
a shaper body having a proximal end and a distal end adapted to engage the bone and tissue in the mammal;
a shaping head operably engaged to the distal end of the shaper body, the shaping head having a first neutral position in line with a central line of the shaper body and a second fully articulated position offset at least 130 degrees from the center line of the shaper body; and
a handle operably engaged to the proximal end of the shaper body adapted to articulate the shaping head from the neutral position to the fully articulated position.

2. The shaper of claim 1 wherein the shaper body includes an actuator operably engaged to the proximal end adapted to incrementally move the shaping head to a fixed position anywhere between the neutral position and the fully articulated position.

3. The shaper of claim 1 wherein the shaping head has a width and the shaper is configured to remove the bone and tissue to create a void at least two times the width of the cutting head.

4. The shaper of claim 1 wherein the shaper has a diameter in the range of about 3 mm to 7 mm.

5. The shaper of claim of 1 wherein the shaping head is configured to move in a sweeping motion.

6. The shaper of claim 1 wherein the shaping head is configured to move in a linear scraping motion.

7. A two stage articulating shaper for use in bone and tissue in a mammal comprising:
a shaper body having a proximal end and a distal end adapted to engage the bone and tissue in the mammal;
a shaping head operably engaged to the distal end of the shaper body, the shaping head having a neutral position in line with the shaper body, a first stage cutting position and a second stage deployment perpendicular from a center line of the shaper body maintaining the first stage cutting position; and
a mechanism adapted to move the shaping head from the neutral position to the first stage cutting position and the second stage deployment position.

8. The shaper of claim 7 wherein the first stage cutting position is between the neutral position and a fully articulated position in the range of about 90 degrees from the neutral position.

9. The shaper of claim 7 wherein the shaping head is configured to maintain any position between the neutral position and the fully articulated position.

10. The shaper of claim 7 wherein the shaping head is configured to move in a sweeping motion.

11. The shaper of claim 7 wherein the shaping head is configured to move in a linear scraping motion.

12. A method of removing bone and tissue from a mammal comprising:
inserting a shaper having a shaping head in a first neutral position in line with a central line of the shaper body into a mammal;
articulating the shaping head from the neutral position to a fully articulated position in a sweeping motion to cyclically sweep out a cavity; and
fixing the shaping head in a desired position;
moving the shaper with the shaping head in a fixed position to scrape the tissue and bone out of the cavity.

13. The method of claim 12 further including the step of:
rotating and/or moving the shaper longitudinally to create the desired cavity shape.

14. The method of claim 12 further including the step of creating a cavity having a width at least two times a width of the shaping head.

15. The method of claim 12 further including the step of:
incrementally rotating the shaper to create the desired cavity shape.

16. A method of providing tools and instructions for removing bone and tissue from a mammal comprising:
providing a shaper comprising
a shaper body having a proximal end and a distal end adapted to engage the bone and tissue in the mammal;
a shaping head operably engaged to the distal end of the shaper body, the shaping head having a first neutral position in line with a central line of the shaper body and a second fully articulated position offset at least 130 degrees from the center line of the shaper body; and
a handle operably engaged to the proximal end of the shaper body adapted to articulate the shaping head from the neutral position to the fully articulated position; and
providing instructions for using the shaper comprising the steps of:
inserting the shaper having a shaper head in the first neutral position in line with a central line of the shaper body into a mammal; and
articulating the shaper head to from the neutral position to the fully articulated position in a sweeping motion to cyclically sweep out a cavity.

17. The method of claim 16 wherein the step of providing instructions further includes the step of:
fixing the shaping head in a desired position to scrape the tissue and bone out of the cavity.

18. The method of claim 16 further wherein the step of providing instructions further includes the step of:
moving the shaper longitudinally to create the desired cavity shape.

19. The method of claim 16 wherein the step of providing instructions further includes the step of:
incrementally rotating the shaper to create the desired cavity shape.

20. The method of claim 16 wherein the step of providing instructions further includes the step of:
creating a cavity having a width at least two times a width of the shaping head.
